(19) United States
(12) Patent Application Publication
(10) Pub. No.: US 2010/0312201 A1
(43) Pub. Date: Dec. 9, 2010

(54) DEVICE FOR APPLYING A FLUID TO A BODY PART

(75) Inventor: Guenther Hoege, Ueberlingen (DE)

Correspondence Address: Muncy, Geissler, Olds & Lowe, PLLC, 4000 Legato Road, Suite 310, FAIRFAX, VA, 22033, US

(73) Assignee: MedoSept AG, Pfaeffikon (CH)

(21) Appl. No.: 12/746,194

(22) PCT Filed: Dec. 4, 2008
(86) PCT No.: PCT/EP08/66831

§ 371 Date: Aug. 23, 2010

(30) Foreign Application Priority Data
Dec. 4, 2007 (DE) 10 2007 058 190.9

(51) Int. Cl. A61M 35/00

(52) U.S. Cl. 604/293

(57) ABSTRACT

The present invention relates, inter alia, to a device for applying a fluid to a body part, in particular to a limb, having a housing having an internal spray chamber, which is accessible through a housing opening for the passage of the body part, wherein said spray chamber has at least one nozzle, from which the fluid is conducted onto the body part, wherein said nozzle is disposed movably relative to said body party received in said spray chamber.

The invention relates to a device for applying a fluid to a body part, in particular to human limbs, such as feet or hands.

In the present case, fluids are to be understood as meaning both pure liquids and gases as well as gas/liquid mixtures or aerosols.

As an alternative to the direct application of a treatment fluid from corresponding storage containers, such as, for example, dispensers or spray cans, onto the body parts to be treated, the prior art discloses a very wide range of devices which provide a spray chamber in which the body part is at least partly held for treatment purposes. The use of a spray chamber is intended to prevent the fluids to be applied from coming into contact with directly surrounding regions, such as floors, walls, clothing, etc instead of with the body part or from entering the surrounding air, in order thus firstly to avoid soiling and secondly to achieve better utilization of the fluid jet or of the fluid stream.

The prior art discloses, for example, a device for applying an aerosol, such as, for example, a spray for athlete's foot, as described in German Utility Model No. 20 2006 015 002 U1. The device has a housing having a spray chamber with a plurality of stationary nozzles, into which the body part onto which the aerosol is to be applied is introduced. If this device is used, for example, for the treatment of the human foot, the user places the foot in the spray chamber in such a way that the sole of the foot comes to rest on the bottom surface of the spray chamber. However, with the use of an aerosol, this means that in particular the region of the sole of the foot and of the heel, namely where the foot rests on the bottom surface of the spray chamber, is not completely exposed to the aerosol. Consequently, not all skin areas are treated during the application, which is undesirable in particular for a treatment with disinfectants.

This is the case even when nozzles are arranged in the region of the bottom surface of the spray chamber, since the user would simply cover these with his foot.

Starting from this, it is the object of the present invention to provide a novel device for applying a fluid to a body part, in particular to human limbs, by means of which it is ensured that the body part can be exposed to the respective treatment fluid over its total surrounding skin area which is to be treated.

This object is achieved in each case by a device for applying a fluid to a body part having the features of claim 1, 6 and 8.

In a first fundamental embodiment, the device comprises a housing having an internal spray chamber which is accessible through a housing opening for the passage of the body part. The spray chamber has at least one spray opening or nozzle from which the fluid is conducted onto the body part. In order to ensure that the total body part can be treated in its entirety, the at least one nozzle is arranged, according to the invention, in the spray chamber so that said nozzle is moveable relative to the body part.

The extent of the relative mobility of the at least one nozzle is chosen so that the total body part held in the spray chamber can be subjected to a treatment with the fluid.

The nozzle may be arranged on a nozzle holder which is guided in a moveable manner within the spray chamber. Both a purely linear and a pivot or rotational movement of the nozzle or of the nozzle holder relative to the limb is conceivable.

Moreover, the at least one nozzle can be arranged in a nozzle holder in the form of a nozzle head which is rotatable per se. Preferably, a plurality of nozzles, optionally of different design, is integrated in such a nozzle head, which nozzles serve for the application of fluids of different consistency or viscosity.

In a particularly advantageous embodiment, the nozzle holder is configured as a sort of fork which is formed and arranged within the spray chamber so that the fork can at least partly rotate with the nozzles around the body part, for example around the foot.

Ideally, a plurality of nozzles is used, where the respective nozzles for the application of a certain fluid can be opposite one another and the fork is arranged in such a way that a complete revolution of the fork is not necessary in order to reach all skin areas of the limb.

If only nozzles arranged in a stationary manner in the spray chamber are used, the invention proposes, in a second fundamental embodiment, such a device for applying a fluid to a body part in which a distance sensor device is provided.

This distance sensor device is configured according to the invention in such a way that triggering of the application of the fluid is activated only when the distance sensor device detects that the body part or the limb is not in contact with the inner walls of the spray chamber at any point. Preferably, this triggering is also effected only when a defined minimum distance between the limb and the inner walls of the spray chamber exists on all sides, in order to guarantee a satisfactory distribution and application of the treatment fluid depending on the arrangement of the nozzles in the spray chamber.

For this purpose, the user, for example, slightly raises the limb present in the spray chamber in order to activate the triggering. The activation of the spray process and hence the holding of the limb in the correct position can then be communicated to the user by an appropriate optical or acoustic signal.

In order for this purpose, for example, to provide the user with easier handling and simpler operation of the device, the device is formed according to the invention so that it is adjustable in its height and/or in its inclination with appropriate orientation of the housing opening in order to be able to adapt to the body sizes and the dimensions of the limbs of the individual users, depending on the relative position of the device to the users.

Both with the use of stationary nozzles and with the use of moveable nozzles, it is always advantageous if the limb within the spray chamber is held on all sides at a sufficient distance from the nozzles.

For this purpose, the invention proposes, in its third fundamental embodiment, that it may be possible to use a holding device for the body part or the limb in the spray chamber. In contrast to the second fundamental embodiment, the user need not himself position the limb within the spray chamber but is provided by means of this third embodiment with aids which assist him, which facilitates the operation and hence the comfort of the device, in particular for elderly people.

The holding device may have a lattice-like or perforated structure with a mesh size or hole distribution such that substantially all skin areas of the limb are accessible to the fluid.

This lattice-like holding device is ideally shaped so that it is at least partly adapted ergonomically to the form of the limb to be treated, which not only increases the comfort for the user but also permits more exact orientation of the limb in the spray chamber relative to the nozzles.

The holding device is anchored in an exchangeable manner in the spray chamber via an appropriate guide and locking device so that the necessary distance from the inner walls of the spray chamber and the nozzles present there is always maintained.

In another configuration, this holding device is provided as a simple rotatable roll which is arranged transversely in the spray chamber and on which the user can support his foot or his hand. During the application of the fluid, the user can easily move his body part back and forth and thus ensures that all skin areas are exposed to the fluid at some time. Ideally, the roll has a lattice-like or perforated lateral surface on which the limb comes to rest, while at least one further nozzle which is directed radially outward can be provided inside the lateral surface in the roll.

In yet another configuration, this holding device is designed in such a way that only by placing therein the body region directly adjacent to the body part region to be treated this region to be treated comes to rest in the spray chamber in a completely freely accessible manner for the stationary and/or moveable nozzles.

This can be realized if a half-open holding collar for, for example, the lower arm or the lower leg can be inserted into the region of the housing opening. The holding collar has an ergonomic design, in particular an inclination relative to the spray chamber, such that the foot or the hand “hangs” completely freely in the spray chamber so that all skin areas are accessible thereby for the fluid to be applied.

In all fundamental embodiments described above, a triggering device by means of which the application of the fluid can be activated according to needs can furthermore be provided.

Such a triggering device can be realized, for example, in the form of a triggering mechanism in that, by placing the body part in the corresponding holding device, whether as ergonomically shaped lattice or as roll, the applied force is mechanically transmitted in order to open the nozzles. However, a contact sensor cooperating with the triggering device is also conceivable.

Alternatively, it is also possible to provide in the spray chamber a noncontact sensor which recognizes when the limb is present completely in the treatment position. In order optionally also to be able to carry out different treatments with different fluids here, it is also possible to provide a sensor device which is capable of detecting which type of limb has just been received in the spray chamber.

According to the invention, it is possible to provide three different nozzles or pairs of nozzles in a stationary and/or moveable arrangement, which serve for respective different treatments of limbs in successive or simultaneous treatment steps.

A first nozzle can be provided for applying an aerosol, such as, for example, a disinfectant. The second nozzle can serve for the application of a liquid, such as, for example, a moisturizing lotion, while the third nozzle can serve for the application of a further liquid of different viscosity, such as, for example, a cosmetic.

All three nozzles can be integrated in a common nozzle head or arranged in an appropriate nozzle holder.

With the use of different fluids, different storage containers are also provided in the housing of the device, preferably in a receptacle accessible from the outside. The storage containers are ideally exchangeable and can be connected to the respective coordinated nozzles via appropriate adapter arrangements and fluid conducting systems.

In order to prevent the emergence of the fluids from the housing opening during the application, the housing opening can have a protective collar which is ergonomically adaptable to the body part and can close the spray chamber thereby. Said protective collar is preferably inflatable in order to place it on the body part.

In addition to the application of fluids by means of the nozzle arrangements, further treatment mechanisms which can be used if required after application of the fluids can be provided within the spray chamber, such as, for example, drying devices by means of blowers or heat radiation or massage devices.

For monitoring or controlling the application process, it is possible to use optical monitoring systems, such as, for example, a UV lamp or another source of ultraviolet radiation having a sensor component tailored thereto. The spectral range which can be used is advantageously tailored here to the fluid to be used. The sensor component detects the body part and recognizes areas which have still not been wetted or not been sufficiently wetted, so that either an inadequate procedure is communicated to the user or the application process is appropriately controlled. The detection or identification of areas which have not been wetted can be effected, for example, by means of scanning methods or image processing, for example by template matching, it being possible for templates of human extremities to be provided or to be recorded or produced by the user. In order to permit the extremities to be distinguished from the background, the latter can be designed to be radiation-absorbing, for example by the use of black surfaces.

Further advantages and features of the invention are evident from the following description of the individual working examples with reference to the attached drawings.

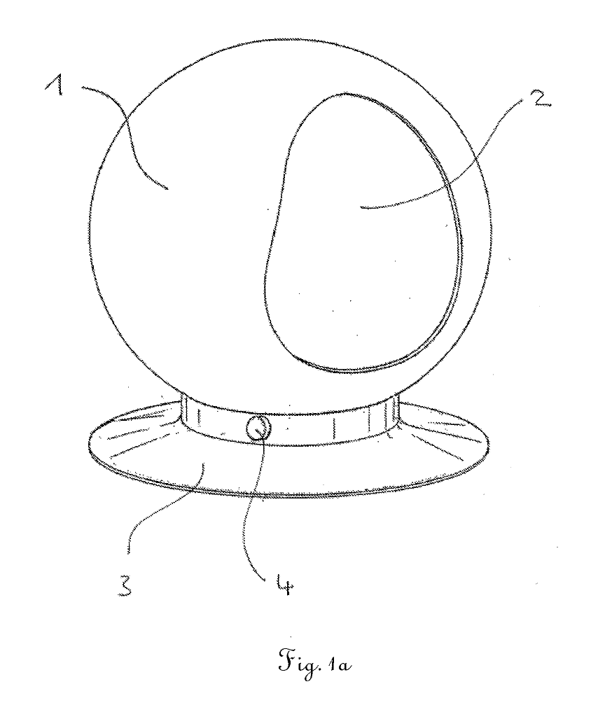

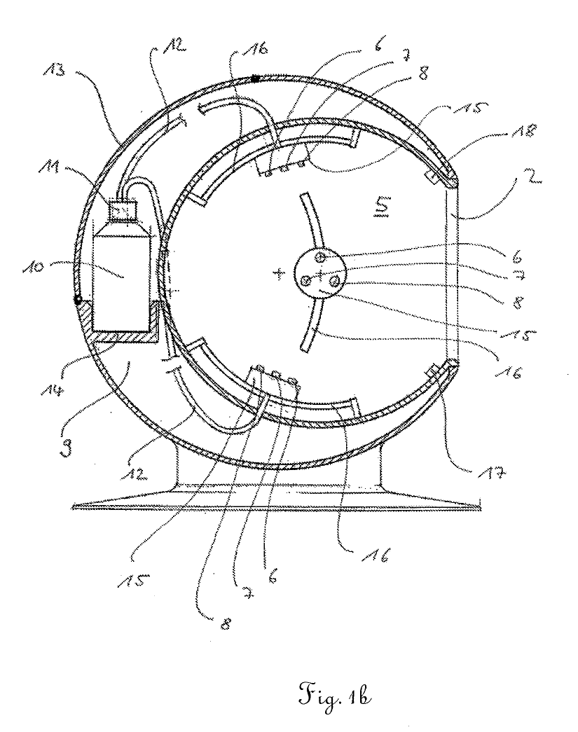

FIG. 1a shows a perspective diagram of a device for applying a fluid to a body part in a first embodiment according to the invention;

FIG. 1b schematically shows the section through the device according to FIG. 1a;

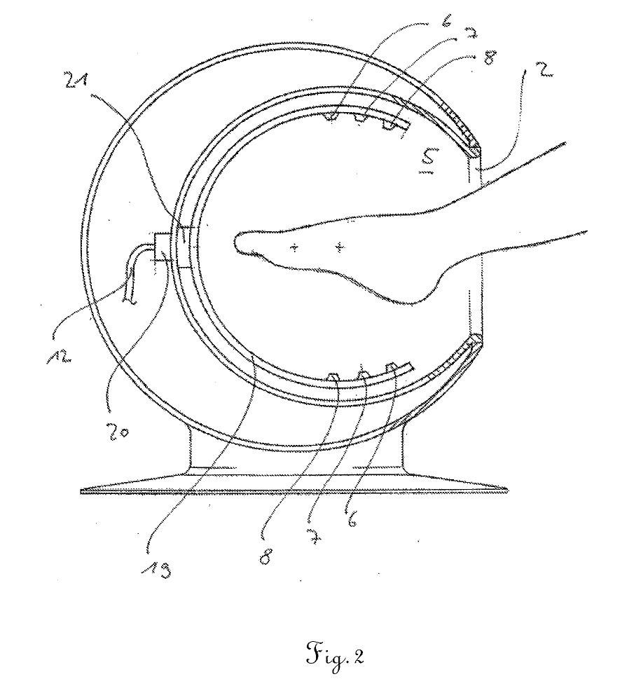

FIG. 2 shows a schematic diagram of a device for applying a fluid to a body part in a second embodiment according to the invention;

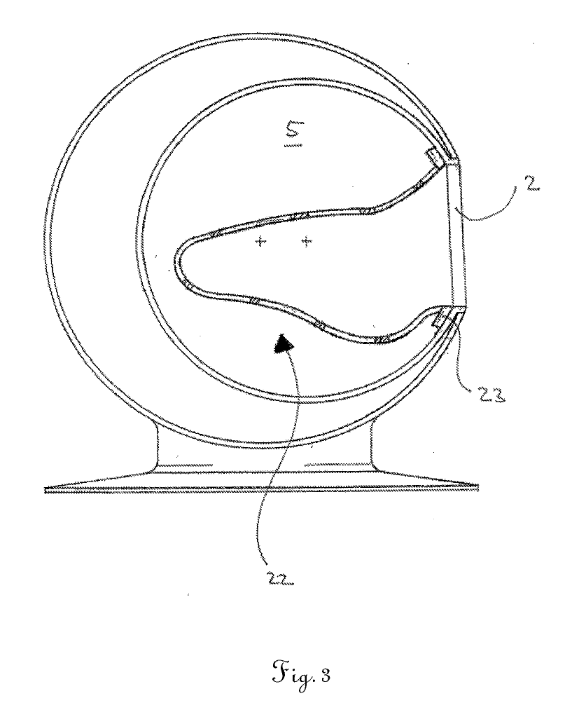

FIG. 3 shows a schematic diagram of a device for applying a fluid to a body part in a third embodiment according to the invention;

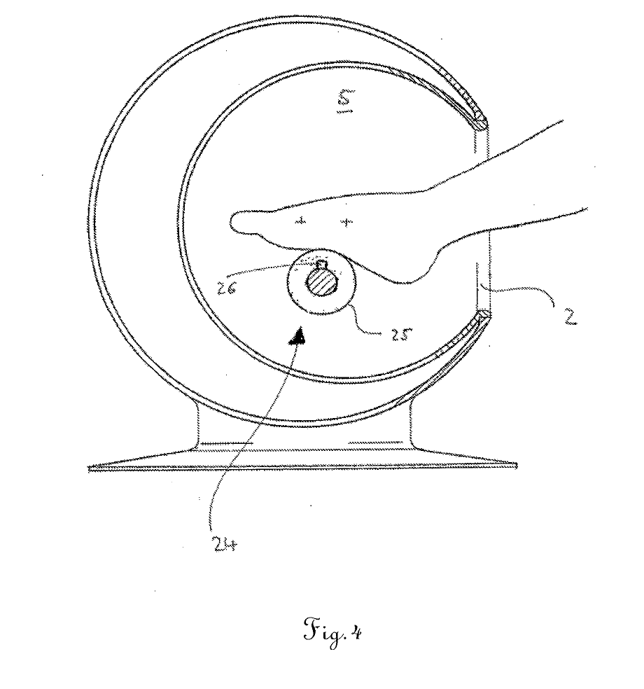

FIG. 4 shows a schematic diagram of a device for applying a fluid to a body part in a fourth embodiment according to the invention;

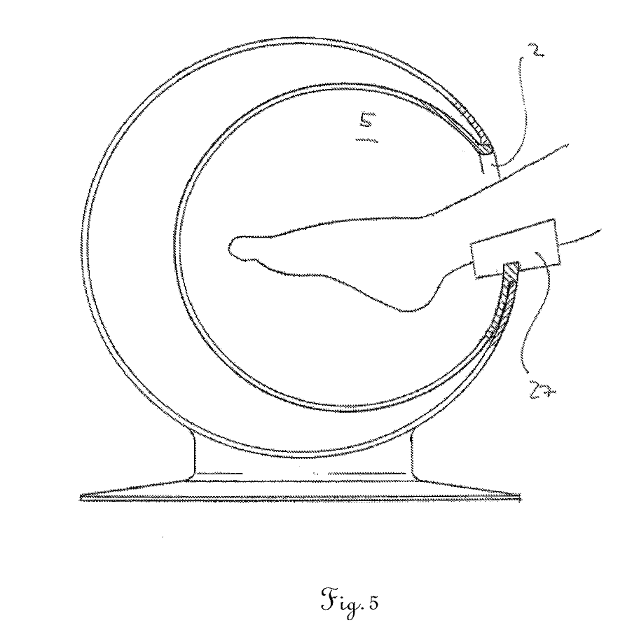

FIG. 5 shows a schematic diagram of a device for applying a fluid to a body part in a fifth embodiment according to the invention; and

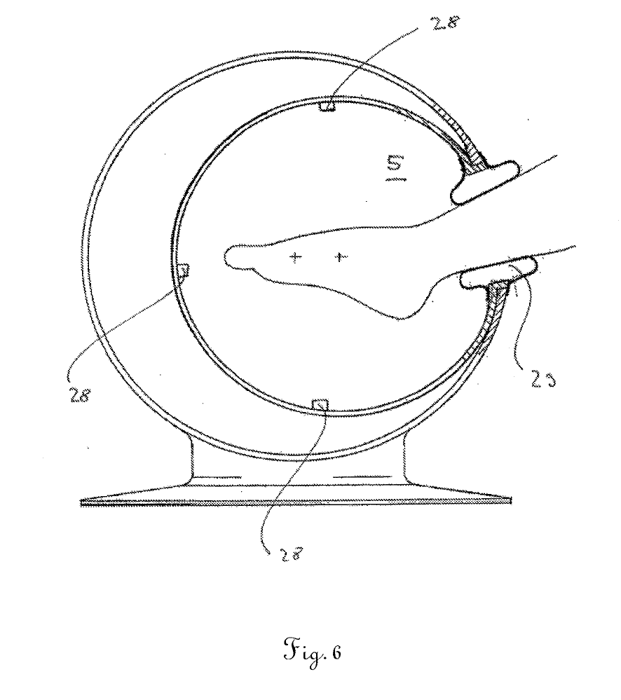

FIG. 6 shows a schematic diagram of a device for applying a fluid to a body part in a sixth embodiment.

FIG. 1a schematically shows a first embodiment of the device for applying a fluid to a body part according to the invention.

The device consists of a housing 1 which has a housing opening 2 through which the body part, for example a hand or a foot, can be introduced for treatment purposes.

The housing 1 is arranged on a base 3 in the form of a stand in such a way that the housing 1 is adjustable in height and/or adjustable in inclination relative to the base 3, with the result that the device and hence the housing opening 2 can be adapted with respect to the user for purposes of better treatment. The height and inclination adjustability can be realized via mechanisms sufficiently well known from industry, such as, for example, telescopic gas springs and the like. By means of a locking mechanism or of a setting device 4, the housing 1 can then be fixed in its position.

FIG. 1b shows the device according to FIG. 1a as a partial section, the individual technical components or features merely being indicated in schematic form.

The housing 1 has a spray chamber 5 which is accessible through the housing opening 2 for the body part to be treated. A plurality of nozzles or pairs of nozzles 6, 7 and 8 are provided in the spray chamber.

At least partly surrounding the spray chamber 5, at least one further receptacle 9 for further components of the device is provided in the housing 1.

This receptacle 9 contains, for example, the storage containers 10 for the individual fluids, only one container being shown here by way of example. By means of an adapter arrangement 11 and a fluid conducting system 12, the individual storage containers 10 are connected to the respective nozzles 6, 7 and 8.

In each case an independent storage container 10 having corresponding fluid conducting systems 12, merely indicated here, is coordinated with each nozzle 6, 7 and 8, so that different treatment fluids can be applied simultaneously or in succession.

The receptacle 9 can be opened from outside through a housing cover 13 in order to create access to the individual components. The storage containers 10 are exchangeably held in a holding device 14. These may be commercially available spray cans containing in each case fluids under pressure. The adapter arrangement 11 is designed so that it permits connection to conventional spray can closures and, if required, can trigger them appropriately.

The nozzles 6, 7 and 8 are arranged in each case on a nozzle holder 15 or a nozzle head. The nozzle holder itself is mounted in a longitudinal displaceable manner on a guide 16. As can be seen, a plurality of such guides 16 are provided in the spray chamber 5, the number and distribution of which guides 16 in the spray chamber 5 are chosen so that complete application of the respective fluids to all skin areas of the limb held in the spray chamber 5 is ensured.

The longitudinal displaceability of the nozzle holders 15 can be effected by suitable motors or pumps not shown here, it being possible to realize a restoration of the nozzle holders 15 by suitable spring means likewise not shown here.

For further support of the moveable nozzles 6, 7 and 8, it is moreover possible to provide in the spray chamber 5 stationary nozzles 17, in particular at points where a moveable mechanism for nozzle holders 15 is not possible owing to a lack of space, for example in the lower region of the housing opening 2, so that, in the treatment of, for example, a foot, the heel region, too, can be completely covered with the fluid.

The device has a sensor device 18, for example an infrared sensor arranged in the spray chamber 5, which detects if the limb to be treated is present in the spray chamber 5 and then appropriately triggers the treatment with the fluid.

FIG. 2 shows a second embodiment of the device according to the invention.

In this embodiment, a rotatably mounted nozzle holder is provided in the spray chamber 5. The nozzle holder 19 is in the form of a fork on which the nozzles 6, 7 and 8 are arranged. The fork moves around a limb, shown here schematically as a foot, during the application of the fluids, a revolution of 180° being sufficient to cover the entire foot.

The fork-like nozzle holder 19 is rotationally driven by a motor 20. Alternatively, it is also conceivable for the bearing 21 of the nozzle holder 19 to be configured so that the nozzle holder 19 is caused to execute a rotational movement by the pressure of the fluid from the fluid conducting system 12 itself, a restoring spring not shown here moving the nozzle holder 19 back to its starting position as soon as the fluid pressure declines.

FIGS. 3 to 5 each show embodiments of the device according to the invention which have corresponding holding devices arranged in the interior of the spray chamber 5, in different configurations.

In these devices, the arrangement of the nozzles can be either exclusively stationary or in the form of moveable nozzles, as was described above with regard to FIGS. 1 and 2.

For purposes of simpler illustration, the features explained above in relation to the embodiments of FIGS. 1 and 2 are omitted in the following FIGS. 3 to 6.

The purpose of the holding devices to be described below lies in the provision of a sufficiently large distance of the body part over its total periphery from the stationary and/or moveable nozzles of the spray chamber, so that the respective fluids can be applied to all skin areas of the limb.

FIG. 3 shows a holding device in the form of a lattice or grid 22. The lattice 22 has a shape ergonomically adapted approximately to the shape of the limb, in this case that of a foot, so that satisfactory positioning and fixing of the foot within the spray chamber 5 is ensured.

The lattice 22 has a mesh size such that virtually all skin areas are accessible for the fluids or become accessible by slight back and forth movement of the limbs. The lattice 22 can be inserted into the spray chamber 5 and can be fixed at appropriate points in the region of the housing opening 2 via guides or snap anchors 23 indicated schematically here.

Owing to the fact that the lattice 22 is exchangeable, a plurality of shapes and sizes of such holding lattices 22 can be provided for different users.

FIG. 4 shows a further embodiment of the device having a different holding device.

This is formed in the spray chamber 5 in the form of a rotatable support roll 24 on which the limb comes to rest. By slight back and forth movement by the user during the application of the fluid, all skin areas of the body part are likewise accessible.

In a further development, the lateral surface 25 of the roll 24 may likewise have a fluid-permeable, lattice-like or perforated structure, it being possible to provide at least one further nozzle 26 inside the roll 24 for application of a fluid, for example to the sole of a foot.

Both the lattice 22 and the roll 24 may have appropriate contact sensors in order to activate triggering of the treatment when the body part rests on the holding device.

FIG. 5 shows yet a further embodiment of the device having a further holding device.

In the housing opening 2, a holding collar 27 can be inserted via appropriate locking mechanisms, for example snap connections not shown here.

This half-open holding collar 27 is ergonomically adapted to the respective body part and has an inclination and orientation such that, by simple placement of the body part, for example of the lower leg in the case of a foot, this can be positioned in the spray chamber 5 so that it rests completely freely from all skin areas to be treated, as shown schematically in FIG. 5. Owing to the fact that the holding collar 27 is exchangeable, a plurality of shapes and sizes of such holding collars 27 can be provided for different users.

FIG. 6 schematically shows a further embodiment of the device according to the invention.

This embodiment is employed exclusively with the use of stationary nozzles or pairs of nozzles.

A plurality of distance sensors 28 which are capable of detecting in each case the distance of the body part introduced into the spray chamber 5 from the inner walls and hence nozzles are distributed in the spray chamber 5. The control is chosen so that the application of the fluid is triggered only in the case of a defined distance of the foot from the respective sensors 28, which the user must reach by briefly raising the foot. This ensures that all skin areas are sufficiently accessible for the fluid.

Furthermore, FIG. 6 shows by way of example a protective collar 29 which can be used in principle on all embodiments.

The protective collar 29 serves for preventing the emergence of fluids during the treatment, in particular in the treatment with an aerosol, from the spray chamber 2.

This protective collar 29 is arranged in the region of the housing opening 2 and designed so that it tightly seals the opening between limb and edge of the housing opening 2 by simple inflation.

In the embodiment according to FIG. 5, the protective collar 29 can also be integrated in the support collar 27.

1. A device for applying a fluid to a body part, in particular to a limb, having a housing having an internal spray chamber which is accessible through a housing opening for the passage of the body part, the spray chamber having at least one nozzle from which the fluid is conducted onto the body part, characterized in that the at least one nozzle is arranged so as to be moveable relative to the body part held in the spray chamber.

2. The device according to claim 1, in which the at least one nozzle is arranged on a moveably arranged nozzle holder.

3. The device according to claim 1, in which the nozzle or the nozzle holder is arranged so as to be linearly or pivotably moveable.

4. The device according to claim 2, in which the nozzle holder is in the form of a fork such that the fork is at least partly moveable around the body part held in the spray chamber.

5. The device according to claim 1, in which a holding device for the body part can be inserted into the spray chamber in such a way that the nozzle or the nozzle holder is moveable around the holding device.

6. A device for applying a fluid to a body part, in particular to a limb, having a housing having an internal spray chamber which is accessible through a housing opening for the passage of the body part, the spray chamber having at least one nozzle from which the fluid is conducted onto the body part, characterized in that a distance sensor device which triggers the application of the fluid only when the body part is not in contact with the inner walls of the spray chamber is provided in the spray chamber.

7. The device according to claim 6, in which the distance sensor device is formed in such a way that the application of the fluid is triggered when the body part is in each case peripherally at a defined distance from the inner walls of the spray chamber.

8. A device for applying a fluid to a body part, in particular to a limb, having a housing having an internal spray chamber which is accessible through a housing opening for the passage of the body part, the spray chamber having at least one nozzle from which the fluid is conducted onto the body part, characterized in that a holding device for the body part can be inserted into the spray chamber.

9. The device according to claim 5, in which the holding device has a lattice-like, fluid-permeable structure.

10. The device according to claim 9, in which the holding device is ergonomically adapted to the body part to be treated.

11. The device according to claim 5, in which the holding device has at least one rotatable roll.

12. The device according to claim 1, in which the lateral surface of the roll has a lattice-like, fluid-permeable structure and the roll has at least one nozzle.

13. The device according to claim 5, in which the holding device is formed in such a way that, by placement of the body part, the body part region which is to be treated rests completely freely in the spray chamber.

14. The device according to claim 1, in which a triggering device by means of which the application of the fluid can be triggered is provided.

15. The device according to claim 14, in which the triggering device has a triggering mechanism or a triggering sensor device.

16. The device according to claim 15, in which the triggering device cooperates with the holding device.

17. The device according to claim 1, in which an exchangeable storage container which can be connected via an adapter arrangement and a fluid conducting system to the nozzle is coordinated with the nozzle in the housing.

18. The device according to claim 1, in which three nozzles or pairs of nozzles are provided in each case for applying different fluids from in each case different exchangeable storage containers.

19. The device according to claim 1, in which a sensor device is provided for detecting the type of body part.

20. The device according to claim 1, in which the device is adjustable in height and/or adjustable in inclination.

21. The device according to claim 1, in which the housing opening has a protective collar which can be ergonomically adapted to the body part and seals the spray chamber.

22. The device according to claim 21, in which the protective collar is inflatable.

23. The device according to claim 1, in which an optical detection device for determining the degree of wetting of the body part is arranged in the spray chamber.

24. The device according to claim 23, in which the optical detection device has a source of ultraviolet radiation.

25. The device according to claim 23, in which the inner wall of the spray chamber is configured to be optically absorbing.
